(19) United States
(12) Patent Application Publication
(10) Pub. No.: US 2025/0349417 A1
(43) Pub. Date: Nov. 13, 2025

(54) TECHNIQUES FOR UTILIZING A MULTI-OUTPUT NEURAL NETWORK

(71) Applicant: Oura Health Oy, Oulu (FI)

(72) Inventors: Gerald Pho, Somerville, MA (US); Xi Zhang, Daly City, CA (US); Tuomas Kentta, Tupos (FI); Raphael Vallat, Lyon (FR)

(21) Appl. No.: 18/661,985

(22) Filed: May 13, 2024

(51) Int. Cl. G16H 40/63; A61B 5/00; A61B 5/0205; A61B 5/024; A61B 5/11; G06F 1/16; G06N 3/08

(52) U.S. Cl. CPC G16H 40/63; A61B 5/0205; A61B 5/6826; G06F 1/163; G06N 3/08; A61B 5/02416; A61B 5/11; A61B 5/7267

(57) ABSTRACT

Methods, systems, and devices for utilizing a multi-output neural network are described. The system may receive physiological data and input the physiological data into the multi-output neural network. The physiological data may include a first input stream corresponding to the heartbeat data and a second input stream corresponding to the motion data. The multi-output neural network is trained to simultaneously compute one or more values of a first physiological metric and one or more values of a second physiological metric. In some cases, the first physiological metric and the second physiological metric each include an input stream from at least one of the first input stream, the second input stream, or both. The system may generate, via a single pass of the multi-output neural network, the one or more values of the first physiological metric and the one or more values of the second physiological metric.

### FIELD OF TECHNOLOGY

The following relates to wearable devices and data processing, including techniques for utilizing a multi-output neural network.

### BACKGROUND

Some wearable devices may be configured to collect data from users associated with heart rate, motion data, temperature data, photoplethysmogram (PPG) data, and the like. In some cases, some wearable devices may perform various actions, such as providing certain health insights to users and based on acquired physiological data in order to assist the user with improving their overall health. However, conventional techniques implemented by wearable devices are deficient.

### BRIEF DESCRIPTION OF THE DRAWINGS

FIG. 1 illustrates an example of a system that supports techniques for utilizing a multi-output neural network in accordance with aspects of the present disclosure.

FIG. 2 illustrates an example of a system that supports techniques for utilizing a multi-output neural network in accordance with aspects of the present disclosure.

FIG. 3 shows an example of a system that supports techniques for utilizing a multi-output neural network in accordance with aspects of the present disclosure.

FIG. 4 shows an example of a system that supports techniques for utilizing a multi-output neural network in accordance with aspects of the present disclosure.

FIG. 5 shows an example of a system that supports techniques for utilizing a multi-output neural network in accordance with aspects of the present disclosure.

FIG. 6 shows a block diagram of an apparatus that supports techniques for utilizing a multi-output neural network in accordance with aspects of the present disclosure.

FIG. 7 shows a block diagram of a wearable device manager that supports techniques for utilizing a multi-output neural network in accordance with aspects of the present disclosure.

FIG. 8 shows a diagram of a system including a device that supports techniques for utilizing a multi-output neural network in accordance with aspects of the present disclosure.

FIG. 9 shows a flowchart illustrating methods that support techniques for utilizing a multi-output neural network in accordance with aspects of the present disclosure.

### DETAILED DESCRIPTION

Wearable devices may be configured to collect physiological data from users to provide users with more information regarding their sleep patterns and overall health. In some cases, such wearable data may be used to detect illnesses or conditions of the user, such as sleep apnea, sleep stages that the user experiences during a night of sleep, blood oxygen saturation (SpO2), blood pressure, and the like. Some wearable devices, however, may utilize separate architectures that independently generate metrics for the detected illnesses and conditions. Because each neural network operates independently of each other, the algorithms may be unable to learn from each other and an increased effort may be used to deploy each of these algorithms on the application. Translating each algorithm into an application compatible language for deployment may be time consuming, error prone, and the like.

Accordingly, aspects of the present disclosure are directed to techniques for using a multi-output neural network that consists of a single neural network architecture that may be utilized for multiple algorithms. Some algorithms use a similar structure such that the predictive algorithms for sleep staging, SpO2, and sleep apnea, for example, may use common inputs (e.g., interbeat intervals (IBIs), motion, temperature data, and the like) and generate outputs with a similar timescale and resolution.

For example, the system may receive the physiological data acquired via the wearable device and input the physiological data into the multi-output neural network. The physiological data may include a first input stream corresponding to the heartbeat data, a second input stream corresponding to the motion data, a third input stream corresponding to temperature data, a fourth input stream corresponding to blood oxygen saturation data, or a combination thereof. The different input streams may be example of the common inputs. The system may generate, via a single pass of the multi-output neural network, one or more values associated with a first physiological metric and one or more values associated with the second physiological metric which may be examples of the generated outputs with a similar timescale and resolution. In such cases, the first physiological metric and the second physiological metric may each include an input stream (e.g., a common input) from at least one of the first input stream, the second input stream, the third input stream, the fourth input stream, or a combination thereof.

The multi-output neural network is trained to simultaneously compute the one or more values associated with the first physiological metric and the one or more values associated with the second physiological metric via the single pass of the multi-output neural network. In such cases, building a single algorithm that may simultaneously handle multiple use cases (e.g., generate outputs for different physiological metrics) may reduce the implementation complexity by maintaining a single architecture (e.g., the multi-output neural network), thereby speeding up the processing time of a computer and other computer resources.

The multi-output neural network may generate the plurality of outputs (e.g., values of the physiological metrics) using a single pass such that the neural network may process the inputs once and return multiple, different outputs. As such, the use of the multi-output neural network speeds up the in-application computation time by avoiding redundancy in input data processing, thereby increasing the efficiency of the system, reducing the overall power consumption, and increasing the overall performance of the system (e.g., decreasing the processing power of a computer). In some cases, using the multi-output neural network for multiple detection algorithms may increase the accuracy by enabling the transfer of knowledge between the different tasks which may positively affect the computation of other detection algorithms.

Aspects of the disclosure are initially described in the context of systems supporting physiological data collection from users via wearable devices. Additional aspects of the disclosure are described in the context of systems. Aspects of the disclosure are further illustrated by and described with reference to apparatus diagrams, system diagrams, and flowcharts that relate to techniques for utilizing a multi-output neural network.

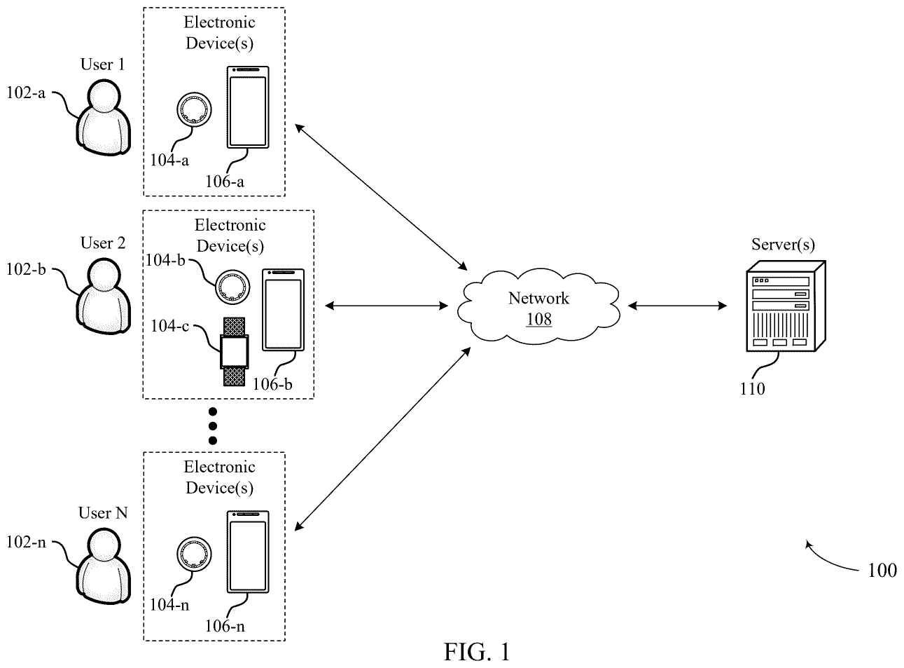

FIG. 1 illustrates an example of a system 100 that supports techniques for utilizing a multi-output neural network in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, SpO2, blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for utilizing a multi-output neural network. The system 100 may include a wearable device 104 configured to acquire physiological data from a user 102. The physiological data may include heartbeat data collected via PPG measurements from one or more light-emitting components and one or more light-receiving components of the wearable device 104, motion data collected via one or more accelerometers of the wearable device, or both. The system 100 may include one or more processors communicatively coupled with the wearable device 104.

The system 100 may use a multi-output neural network that uses specific wearable-based features/measurements as inputs and outputs physiological metrics that are distinct from each other but use the specific wearable-based features/measurements as common inputs. For example, the system 100 may receive the physiological data acquired via the wearable device 104 via one or more electronic signals and input the physiological data into the multi-output neural network. In some cases, the physiological data inputted into the multi-output neural network may include a first input stream corresponding to the heartbeat data and a second input stream corresponding to the motion data.

The multi-output neural network is trained to simultaneously compute one or more values associated with a first physiological metric and one or more values associated with a second physiological metric where the first physiological metric and the second physiological metric each include an input stream from at least one of the first input stream, the second input stream, or both. The system 100 may generate, via a single pass of the multi-output neural network, the one or more values associated with the first physiological metric and the one or more values associated with the second physiological metric. In some cases, the system 100 may transmit an instruction, to a GUI of a user device 106 associated with the wearable device 104. The instruction is configured to cause the GUI to display one or more messages.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

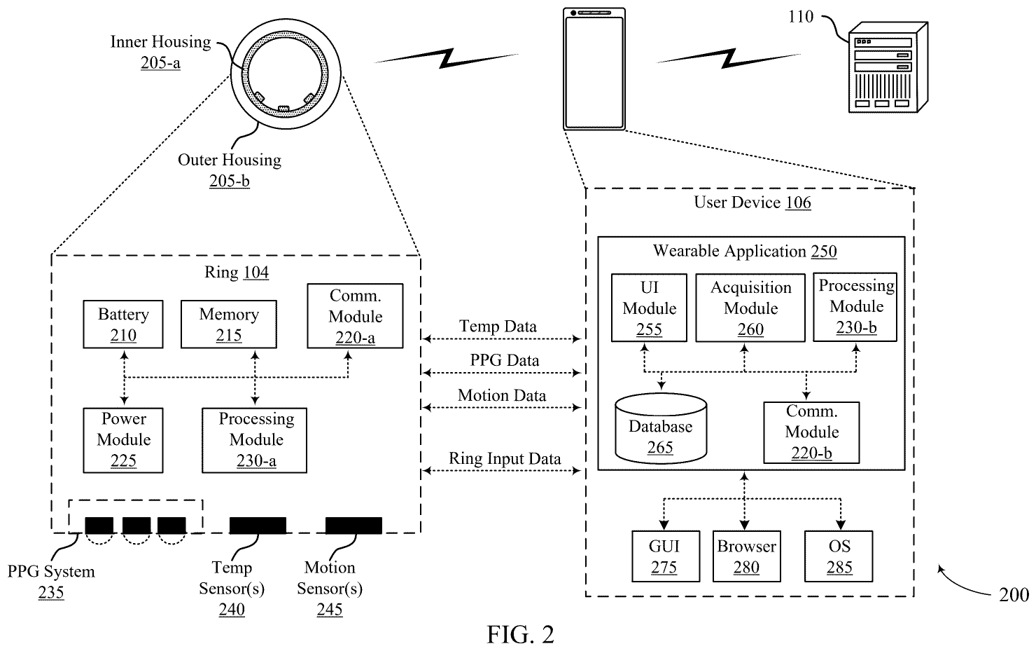

FIG. 2 illustrates an example of a system 200 that supports techniques for utilizing a multi-output neural network in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an IBI. The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros).

The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for utilizing a multi-output neural network. The system 200 may include a wearable device (e.g., ring 104) configured to acquire physiological data from a user. The physiological data may include heartbeat data and blood oxygen data collected via PPG measurements from one or more light-emitting components and one or more light-receiving components of the wearable device (e.g., PPG system 235), motion data collected via one or more accelerometers of the wearable device (e.g., motion sensors 245), temperature data collected via one or more temperature sensors of the wearable device (e.g., temperature sensors 240), or a combination thereof.

The system 200 may include one or more processors (e.g., processing module 230-a) communicatively coupled with the wearable device and configured to receive the physiological data acquired via the wearable device via one or more electronic signals. The one or more processors may be coupled within the wearable device or external to the wearable device (e.g., coupled within a user device associated with the wearable device). The system 200 may input the physiological data into the multi-output neural network. The physiological data may include at least a first input stream corresponding to the heartbeat data. The physiological data may additionally include a second input stream corresponding to the motion data, a third input stream corresponding to the temperature data, a fourth input stream corresponding to the blood oxygen data, or a combination thereof.

The multi-output neural network is trained to simultaneously compute one or more values associated with a first physiological metric and one or more values associated with a second physiological metric. The first physiological metric is associated with a first physiological phenomenon and the second physiological metric is associated with a second physiological phenomenon that is distinct from the first physiological phenomenon. The first physiological metric and the second physiological metric may include at least one of a breathing disturbance event, a sleep staging metric, a blood oxygen metric, a blood pressure metric, a heartbeat metric, or a respiratory rate metric. The first physiological metric and the second physiological metric may include an input stream from the first input stream and at least one of the second input stream, the third input stream, the fourth input stream, or a combination thereof.

The system 200 may generate, via a single pass of the multi-output neural network, the one or more values associated with the first physiological metric and the one or more values associated with the second physiological metric. In some cases, the system 200 may transmit an instruction to a GUI of a user device 106 associated with the wearable device based on generating the one or more values associated with the first physiological metric and the one or more values associated with the second physiological metric.

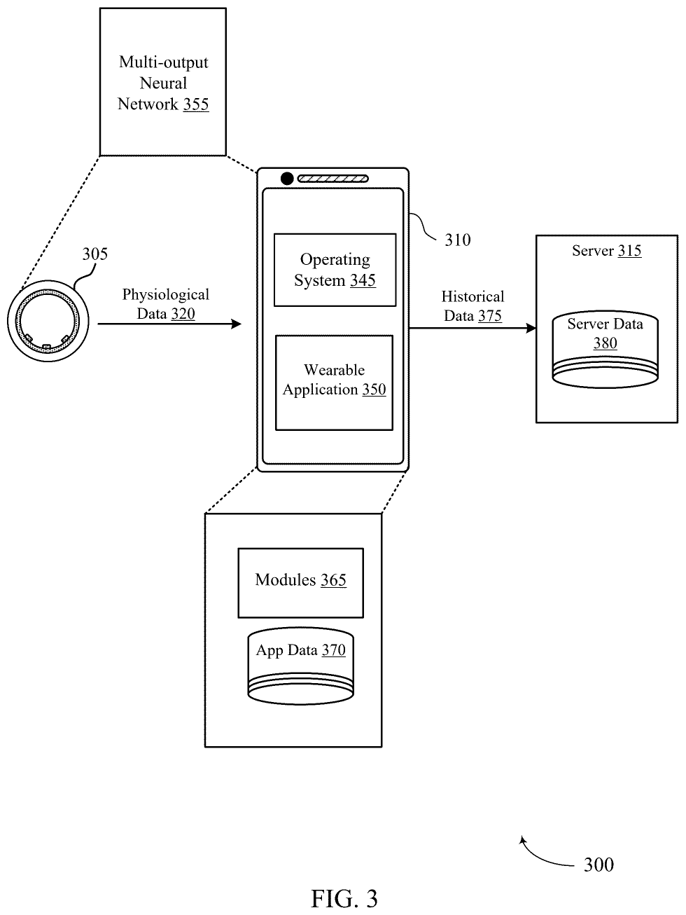

FIG. 3 shows an example of a system 300 that supports techniques for utilizing a multi-output neural network in accordance with aspects of the present disclosure. The system 300 may implement, or be implemented by, system 100, system 200, or both. In particular, system 300 illustrates an example of a ring 305 (e.g., wearable device 104), a user device 310, and a server 315, as described with reference to FIG. 1.

The ring 305 may acquire physiological data 320. The physiological data 320 may include temperature data, heart rate data, respiratory rate data, HRV data, sleep data, blood oxygen data, heartbeat data, motion data, skin conductance data, pH data, blood glucose data, salt data, among other forms of physiological data as described herein. The ring 305 may transmit physiological data 320 to the user device 310. The temperature data may include continuous nighttime temperature data. The respiratory rate data may include continuous nighttime breath rate data. In some cases, multiple devices may acquire physiological data 320. For example, a first computing device (e.g., user device 310) and a second computing device (e.g., the ring 305) may acquire the physiological data 320.

The ring 305 (e.g., wearable device) may be configured to acquire physiological data 320 from a user. The heartbeat data and SpO2 data may be collected via PPG measurements from one or more light-emitting components and one or more light-receiving components of the ring 305. The motion data may be collected via one or more triaxial accelerometers of the wearable device. The temperature data may be collected via one or more temperature sensors.

For example, the ring 305 may acquire user physiological data 320, such as temperature data, respiratory rate data, heart rate data, HRV data, sleep data, motion data, SpO2 data, (e.g., blood oxygen saturation data), heartbeat data, galvanic skin response, actigraphy, and/or other user physiological data. The ring 305 may acquire raw data and convert the raw data to features with daily granularity. In some implementations, different granularity input data may be used. The ring 305 may send the data to another computing device, such as a mobile device (e.g., user device 310) for further processing. In some cases, the system 300 may smooth the data (e.g., using a 7-day smoothing window, a 90-day smoothing window, or other window). The missing values may be imputed (e.g., using the forecaster Impute method from the python package).

Although the system may be implemented by a ring 305 and a user device 310, any combination of computing devices described herein may implement the features attributed to the system 300. For example, the wearable device configured to acquire the physiological data may be an example of the ring 305, a wrist-worn device, a neck-worn device, or a combination thereof. As described herein, the multi-output neural network 355 may be included in the ring 305, the user device 310, or both.

The user device 310 may include the wearable application 350 and an operating system 345. The wearable application 350 may run on the operating system 345 of a user device 310 and is associated with a ring 305. The wearable application 350 may include at least modules 365 and application data 370. In some cases, the application data 370 may include historical physiological data patterns for the user and other data. The physiological data patterns may include temperature data, heart rate data, respiratory rate data, HRV data, sleep data, blood oxygen saturation data, heartbeat data, motion data, or a combination thereof.

Machine-learning pipelines (e.g., neural networks) may process and leverage large amounts of data, as well as exploit similarities across different problem areas. Once an algorithm is ready for deployment and use, the system 300 may desire to deploy the algorithm with increased speeds and efficiency and with minimum technical burden.

In previous versions of the system 300, each algorithm of the system 300 may be independent of each other. In such cases, each algorithm may utilize different inputs and machine learning models. As a consequence, a large effort may be used to deploy these algorithms on the wearable application 350 because each algorithm is implemented separately. In such cases, the algorithms may be unable to learn from each other, thereby limiting the training accuracy of each algorithm.

In previous versions of the system 300, the algorithms may be built using heuristic, simple machine learning (e.g., a linear algorithm with smaller data sends) and/or using standard Python machine-learning and data science stack (i.e., pandas, numpy, sklearn, LightGBM, etc), and therefore may be translated into an application-compatible language for deployment which may be prone to errors and time consuming. In some cases, the system 300 may maintain multiple copies of the same algorithm. In some examples, algorithms in previous versions of the system 300 may not use deep neural networks.

As the amount of training data increases, deep learning algorithms may produce better results than traditional machine learning algorithms, such as tree-based models. In such cases, an increased amount of data within the system 300 (e.g., including at least the continuously acquired physiological data 320), may enable the system 300 to train deep neural network models (e.g., a multi-output neural network 355) that may complete several tasks at once, thereby generating multiple outputs from a single pass of the multi-output neural network 355, as described herein.

Techniques may be described herein to utilize a multi-output neural network 355. In such cases, a single network architecture may be utilized for many multiple algorithms within the system 300. The architecture may be modular and easily reused with minimal technical effort for implementation in future algorithms. Utilizing a multi-output neural network 355 may enable the system 300 to utilize a single neural network that uses multiple inputs and generates multiple outputs with a single-pass of the neural network, thereby reducing the implementation complexity by maintaining a single architecture and/or model, increasing accuracy by enabling transfer of knowledge between different tasks, and increasing the speeds associated with in-application computation time by avoiding redundancy in input data processing.

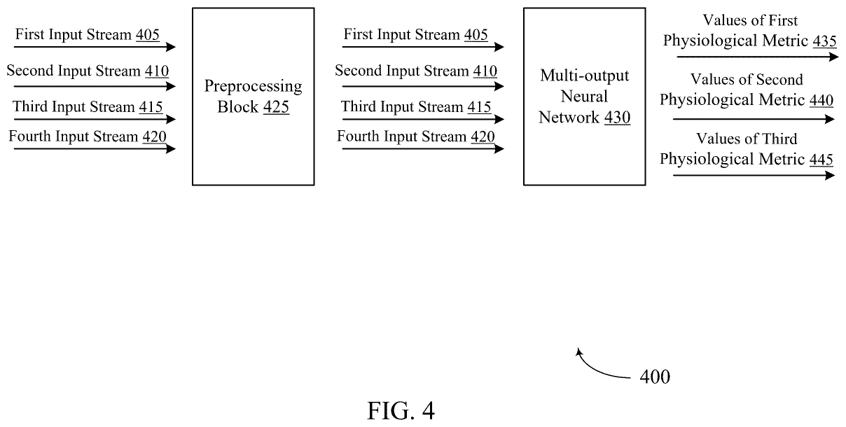

The user device 310 may include the multi-output neural network 355. In such cases, the wearable application 350 may include the multi-output neural network 355. The one or more processors of the user device 310 may receive the physiological data 320 acquired via the ring 305 via one or more electronic signals. The processors may input the physiological data 320 into the multi-output neural network 355. The multi-output neural network 355 is trained to simultaneously compute one or more values associated with a first physiological metric and one or more values associated with a second physiological metric. The first physiological metric and the second physiological metric each include an input stream from at least one of a first input stream, a second input stream, a third input stream, a fourth input stream, or any combination thereof, as described with reference to FIG. 4.

The processors of the user device 310 may generate, via a single pass of the multi-output neural network 355, the one or more values associated with the first physiological metric and the one or more values associated with the second physiological metric. The processors may transmit an instruction to a GUI of a user device 310 associated with the ring 305 to display one or more messages in response to generating the one or more values associated with the first physiological metric and the one or more values associated with the second physiological metric.

The wearable application 350 may present one or more messages associated with the one or more values associated with the first physiological metric and the one or more values associated with the second physiological metric to the user. The wearable application 350 may include an application data processing module that may perform data processing. For example, the application data processing module may include modules 365 that provide functions attributed to the system 300. The wearable application 350 may store application data 370, such as acquired physiological data 320.

The system 300 may cause a GUI of the user device 310 to display the one or more messages associated with the one or more values associated with the first physiological metric and the one or more values associated with the second physiological metric. The system 300 may generate a message for display on a GUI on the user device 310 that indicates the one or more values associated with the first physiological metric and the one or more values associated with the second physiological metric. The generation of the one or more values associated with the first physiological metric and the one or more values associated with the second physiological metric may trigger a personalized message to a user highlighting the educational content associated with the first physiological metric and/or the second physiological metric. In some cases, the message may include a recommendation to rest, recommendations to improve athletic performance, a recommendation to exercise, an adjusted set of sleep targets, a quantity of minutes in deep sleep, a recommended wake time for the user, a recommended bedtime for the user, or a combination thereof.

In some implementations, the wearable application 350 may notify the user of the one or more values associated with the first physiological metric and the one or more values associated with the second physiological metric and/or prompt the user to perform a variety of tasks in the activity GUI. The notifications and prompts may include text, graphics, and/or other user interface elements. The notifications and prompts may be included in the wearable application 350 such as when the one or more values associated with the first physiological metric and the one or more values associated with the second physiological metric are generated, the wearable application 350 may display notifications and prompts. The user device 310 may display notifications and prompts in a separate window on the home screen and/or overlaid onto other screens (e.g., at the very top of the home screen). In some cases, the user device 310 may display the notifications and prompts on a mobile device, a user's watch device, or both.

In some implementations, the user device 310 may store historical user data 375. The historical data 375 may include historical temperature patterns of the user, historical heart rate patterns of the user, historical respiratory rate patterns of the user, historical HRV patterns of the user, historical sleep data, historical blood oxygen saturation of the user, historical motion data of the user, historical heartbeat data of the user, or a combination thereof. The historical data 375 may be selected from the last few months. The historical data 375 may be used by the server 315. Using the historical data 375 may allow the user device 310 and/or server 315 to personalize the GUI by taking into consideration the user's historical data 375.

The user device 310 may transmit historical data 375 to the server 315. In some cases, the transmitted historical data 375 may be the same historical data stored in the wearable application 350. In other examples, the historical data 375 may be different than the historical data stored in the wearable application 350. The server 315 may receive the historical data 375. The server 315 may store the historical data 375 in server data 380.

In some implementations, the user device 310 and/or server 315 may also store other data that may be an example of user information. The user information may include, but is not limited to, user age, weight, height, body mass index, gender, phone audio, and medical history of the user. In some implementations, the user information may be used as input streams for computing one or more values associated with a first physiological metric and one or more values associated with a second physiological metric. The server data 380 may include the other data such as user information.

FIG. 4 shows an example of a system 400 that supports techniques for utilizing a multi-output neural network in accordance with aspects of the present disclosure. The system 400 may include a plurality of input streams, a preprocessing block 425, a multi-output neural network 430, and a plurality of outputs (e.g., values of characteristics), which may be examples of the input streams, the multi-output neural network 430, and values of characteristics as described with reference to FIGS. 1 through 3.

The system 400 may input the input streams into the preprocessing block 425. The input streams may include a first input stream 405, a second input stream 410, a third input stream 415, and a fourth input stream 420. In some cases, the preprocessing block 425 may receive one or more input streams including, but not limited to, any combination of a first input stream 405, a second input stream 410, a third input stream 415, and a fourth input stream 420.

The first input stream 405 may be an example of heartbeat data, the second input stream 410 may be an example of motion data, the third input stream 415 may be an example of temperature data, and the fourth input stream 420 may be an example of blood oxygen data. In some cases, the preprocessing block 425 may receive other input streams such as audio input, user information (e.g., gender, age, weight, BMI, and the like), respiratory rate data, HRV data, and the like.

The preprocessing block 425 may perform a single preprocessing step on the input data (e.g., the input streams). In such cases, the first input stream 405, the second input stream 410, the third input stream 415, and the fourth input stream 420 may be preprocessed simultaneously prior to inputting the input streams into the multi-output neural network 430. The preprocessing block 425 may output the preprocessed input streams and input the preprocessed input streams into the multi-output neural network 430.

The multi-output neural network 430 may receive at least the first input stream 405. In some cases, the multi-output neural network 430 may receive the first input stream 405 and any combination of the second input stream 410, the third input stream 415, the fourth input stream 420. The multi-output neural network 430 is trained to simultaneously compute one or more values associated with a first physiological metric 435, one or more values associated with a second physiological metric 440, one or more values of a third physiological metric 445, or any combination thereof using at least one the first input stream 405 and any combination of the second input stream 410, the third input stream 415, the fourth input stream 420.

The first physiological metric, the second physiological metric, and the third physiological metric may each include an input stream from at least one of the first input stream 405, the second input stream 410, the third input stream 415, the fourth input stream 420, or any combination thereof. For example, the first physiological metric, the second physiological metric, and the third physiological metric may each include the first input stream 405 used to generate the one or more values associated with a first physiological metric 435, one or more values associated with a second physiological metric 440, and one or more values of a third physiological metric 445.

In such cases, the system 400 may generate, via a single pass of the multi-output neural network 430, the one or more values associated with a first physiological metric 435, one or more values associated with a second physiological metric 440, and one or more values of a third physiological metric 445. The system 400 may perform a single pass of the multi-output neural network 430 to output multiple values for multiple characteristics, which may save processing power, time, and resources as compared to performing separate passes in separate algorithms. In some cases, the multi-output neural network 430 may perform a single forward pass that is used to generate the values of the first physiological metric 435, the values of the second characteristic, and the values of the third physiological metric 445. That is, the ring input data may be processed once, yet more than one different output may be returned.

The first physiological metric may be associated with a first physiological phenomenon and the second physiological metric may be associated with a second physiological phenomenon that is distinct from the first physiological phenomenon. The third physiological metric may be associated with a third physiological phenomenon that is distinct from both the first physiological phenomenon and the second physiological phenomenon. For example, at least one of the first physiological metric, the second physiological metric, and the third physiological metric may include at least one of a breathing disturbance metric, a sleep staging metric, a blood oxygen metric, a blood pressure metric, a heartbeat metric (e.g., arrhythmia metric), or a respiratory rate metric.

For example, the one or more values of the first physiological metric 435 may be an example of a duration of time that the user experiences deep sleep, a recommended bedtime for the user, a duration of time that user experiences light sleep, and the like for a sleep staging metric (e.g., the first physiological metric). In some examples, the one or more values of the second physiological metric 440 may be an example of a quantity of breathing disturbance events the user experienced during the night, a quantity of times that the user experiences a breathing disturbance event, and the like for the breathing disturbance metric (e.g., the second physiological metric).

Breathing disturbance events (e.g., sleep apnea, sleep hypopnea, breathing disturbance, oxygen saturation, respiratory effort-related arousal (RERA) may result in one or more physiological changes in a user. For example, a breathing disturbance event may cause features such as a decrease in SpO2 of the user, a decrease in body temperature (e.g., skin temperature) of the user, a decrease in pulse wave amplitude (e.g., a volume of blood in the tissue of the user), an increase and/or decrease in heart rate (e.g., IBI) of the user, an intensity of motion of the user to increase, and an ambient noise level to increase (e.g., due to snoring). In some examples, the pulse wave amplitude of the user may decrease as blood vessels of the user constrict due to a decrease in oxygen (e.g., SpO2). In some examples, the heart rate of the user may decrease at a beginning of an apnea-related event and increase at an end of an apnea-related event (e.g., due to apnea-related arousal).

Accordingly, the user device may use any combination of features described herein (e.g., including the multi-output neural network 430) to determine if a user is experiencing a breathing disturbance event. For example, the user device may trigger the wearable device to acquire physiological data (e.g., via SpO2 measurements, other PPG measurements such as IBI and pulse wave amplitude, temperature measurements, motion measurements, and/or audio measurements) upon detecting that the user is asleep (e.g., or that the user is in a certain stage of sleep). The user device may input some or all of the physiological data (e.g., the input streams) into the multi-output neural network 430, which may enable the user device to determine if the user experienced a breathing disturbance event. As described herein, the physiological data may be a time series of physiological data collected during the sleep period of the user. Each input parameter (e.g., each type of measurement) may be associated with a same or different sampling frequency and a uniform or irregular sampling rate.

In some cases, the sleep algorithms may share a similar structure where each of the sleep staging metric, breathing disturbance metric, and blood oxygen metric may use the same ring inputs (e.g., IBIs, motion, temperature, and the like), and process the data at different timescales across the entire night to produce a time-series output. In such cases, it may be desired to utilize a single algorithm that can handle all of these use cases simultaneously. The multi-output neural network 430 may unlock new capabilities in calculating a breathing disturbance metric, improve existing calculations for the sleep staging metric and blood oxygen metric algorithms, and reduce the time and effort required for productization. In some cases, the multi-output neural network 430 may be used for the development of new sleep algorithms (e.g., nighttime blood pressure, nighttime atrial fibrillation, and the like).

The system 400 may utilize the multi-output neural network 430 to complete multiple tasks. For example, the single, multi-output neural network 430 may generate multiple different outputs at the same time using the same or similar inputs. In such cases, the multi-output neural network 430 may generate different, parallel outputs streams (e.g., values of the characteristics). The multi-output neural network 430 may use a shared set of features that enables the multi-output neural network 430 to be better at solving different tasks through the use of a single model using the shared set of features (e.g., inputs streams). The multi-output neural network 430 may choose to use certain tasks more for one feature than another. In such cases, the multi-output neural network 430 may use the same inputs for multiple different tasks.

For example, the multi-output neural network 430 may compute three different, separate outputs (e.g., values of the first physiological metric 435, values of the second physiological metric 440, and values of the third physiological metric 445). The inputs (e.g., input streams) for the outputs may be the same (e.g., including at least the first input stream 405, the second input stream 410, the third input stream 415, the fourth input stream 420, or a combination thereof). In such cases, each of the outputs may be generated using the same inputs.

The outputs of the multi-output neural network 430 may be examples of probabilities. For example, the first physiological metric may be associated with a probability that the user experienced the first physiological phenomenon during a time period, the second physiological metric is associated with a probability that the user experienced the second physiological phenomenon during the time period, and the third physiological metric is associated with a probability that the user experienced the third physiological phenomenon during the time period. In some examples, the first physiological metric may be an example of a probability that the user experienced deep sleep, and the second physiological metric may be an example of a probability that the user experienced a breathing disturbance event.

The outputs of the multi-output neural network 430 may have a common time scale. For example, the one or more values associated with the first physiological metric 435 include a first time scale and the one or more values associated with the second physiological metric 440 may include a second time scale same as the first time scale. In some cases, the second time scale may be different than the first time scale. The one or more values associated with the third physiological metric 445 may include a third time scale that is the same or different than the first time scale, the second time scale, or both. The time scale may be an example of a quantity of minutes, hours, days, a time of day, a day of the week, a sleeping period, a resting period, an awake period, or any combination thereof.

In some examples, each of the one or more values associated with the first physiological metric 435, the one or more values associated with the second physiological metric 440, and the one or more values associated with the third physiological metric 445 may be an example of a time-series with a thirty second resolution (e.g., time scale) that starts at the beginning of the detected sleep period and ends at the end of the detected sleep period.

In one example, the multi-output neural network 430 may output a probability time series indicating a probability that the user experienced a breathing disturbance event during one or more time periods (e.g., timestamps) during the sleep period of the user. An output time resolution associated with the multi-output neural network 430 may be one or more seconds, minutes, hours, and the like. In some examples, the multi-output neural network 430 may output a single probability (e.g., a probability of whether the user experienced a breathing disturbance event at any point during the sleep period).

The system 400 may binarize the probability time series. For example, the system 400 may determine, for each timestamp, whether the user experienced a breathing disturbance event. The system 400 may binarize the probability time series by comparing the probability of each time stamp to a threshold. For example, if the probability associated with a timestamp is greater than a threshold, the system 400 may determine that the user experienced a breathing disturbance event during the timestamp. If the probability associated with a timestamp is less than a threshold, the system 400 may determine that the user did not experience a breathing disturbance event during the timestamp. The probability time series or the binarized probability time series may be referred to herein as the breathing disturbance metric.

In some examples, the system 400 may generate information related to the breathing disturbance metric, such as an index (e.g., an apnea-hypopnea index (AHI), a hypoxic burden (HB) a breathing disturbance index (BDI), an oxygen desaturation index (ODI)) or a timeline of breathing disturbance during the sleep period. In some aspects, the AHI may represent an average quantity of apneas (e.g., complete or almost complete cessation of airflow) and hypopneas (e.g., partial reduction in airflow) experienced by the user per hour of sleep. Mild apnea may correspond to an AHI between 5 and 15, moderate apnea may correspond to an AHI between 15 and 30, and severe apnea may correspond to an AHI over 30. The ODI may represent an average quantity of times per hour that the user's SpO2 decreased by a certain percentage (e.g., from a baseline SpO2 of the user), such as 3 or 4 percent. The HB may represent a total area under an oxygen saturation curve (e.g., relative to a baseline oxygen saturation), which may provide a metric of a severity of oxygen desaturation in individuals with obstructive sleep apnea. A higher index (e.g., AHI, ODI, HB, or BDI) may indicate that the user experienced a relatively greater quantity of breathing disturbance events than a lower index. The system 400 may cause a GUI to display the index and/or the timeline via a GUI of the system 400.

In some examples, the system 400 may determine (e.g., calculate) a breathing quality metric (e.g., a breathing Score). The breathing quality metric may be an inverse of the index, such that a relatively higher breathing quality metric is indicative of relatively fewer breathing disturbance events, a relatively higher average SpO2, a relatively higher pulse wave amplitude, and so on than a relatively lower breathing quality metric. The user device may display the breathing quality metric via the GUI.

In some examples, the process of acquiring physiological data and generating the breathing disturbance metric and/or index may be repeated one or more times (e.g., during one or more sleep periods or nights that the user wears the wearable device). Accordingly, the user may acquire continuous and extended estimates of breathing disturbance severity over time. The system 400 may average breathing disturbance metric data collected over multiple sleep periods of the user (e.g., multiple days) to provide a more robust assessment of sleep apnea severity as compared to data collected during a single sleep period. Such longitudinal monitoring of breathing disturbance may allow the system 400 to assess an effectiveness of interventions or lifestyle changes (e.g., changes in eating or drinking habits, changes in sleep habits, medication) on the breathing disturbance of the user. The user may therefore use personalized and proactive management strategies to decrease a risk of breathing disturbance events.

In some cases, the multi-output neural network 430 may compute one of the physiological metrics that may positively affect computation of the other physiological metrics. In such cases, a training accuracy associated with training the multi-output neural network 430 to compute the one or more values of the first physiological metric 435 is increased based on the multi-output neural network 430 being trained to compute the one or more values of the second physiological metric 440, the one or more values of the third physiological metric 445, or both. The accuracy improvement may occur during training of the model weights rather than during the single pass of computation.

For example, the multi-output neural network 430 may use the breathing disturbance metric to compute the sleep staging metric and vice versa. If the multi-output neural network 430 knows about the breathing disturbance metric, it may not help the multi-output neural network 430 directly compute the sleep staging metric, but it helps because the multi-output neural network 430 knows more about the truth of the data about the user's physiological data included in the input streams. In such cases, predictions of the different characteristics may help each other. Using the multi-output neural network 430 for multiple detection algorithms may increase the accuracy by enabling the transfer of knowledge between the different tasks (e.g., knowing when a breathing disturbance event occurs may benefit sleep staging metrics and vice versa). In such cases, having a common feature set (e.g., common input streams) may help the multi-output neural network 430 learn from each input stream.

The system 400 may continuously train the multi-output neural network 430 based on the inputs (e.g., input streams) and outputs (e.g., values of the characteristics). For example, the system 400 may train the multi-output neural network 430 based on inputting the physiological data into the multi-output neural network 430 and generating the one or more values associated with the first physiological metric 435 and the one or more values associated with the second physiological metric 440. In some cases, the system 400 may train the multi-output neural network 430 based on a training physiological dataset. In such cases, the system 400 may train the multi-output neural network 430 based on a plurality of features within a training physiological dataset associated with a plurality of users.

The training physiological dataset may include physiological data acquired from one or more users and may include ground-truth labels that are indicative of the one or more users experiencing or not experiencing one of the physiological phenomena, such as a breathing disturbance event, for example. For example, a ground-truth label of the training physiological dataset may include a scoring (e.g., a human-expert scoring) of whether a physiological measurement is indicative of a breathing disturbance event. The ground-truth labels may therefore indicate whether the physiological dataset is indicative of breathing disturbance events (e.g., apnea, hypopnea, oxygen desaturation, RERA) throughout a sleep period (e.g., a night), as measured using polysomnography.

The multi-output neural network 430 may further include one or more parameters or weights. By training the multi-output neural network 430 on the training physiological dataset, the system 400 may determine values of the one or more characteristics. For example, the system 400 may iteratively learn to decrease (e.g., minimize) an error between predicted labels (e.g., an output of the multi-output neural network 430 using the training physiological dataset) and the ground-truth labels. Thus, training the multi-output neural network 430 may increase an accuracy of the multi-output neural network 430 in determining whether a set of physiological data is indicative of one of the physiological phenomenon.

The multi-output neural network 430 may be an example of a machine learning model that includes a single executable file that can be directly loaded from within the mobile application. The multi-output neural network 430 may use a convolutional neural network that outperforms traditional machine learning methods. Utilizing the multi-output neural network 430 may enable the multi-output neural network 430 to be easily transferred from a web-based application to a mobile-based application through a language that may be compiled for use on a user device. The multi-output neural network 430 may learn different tasks at once. The multi-output neural network 430 may result in higher accuracy outputs as compared to other systems.

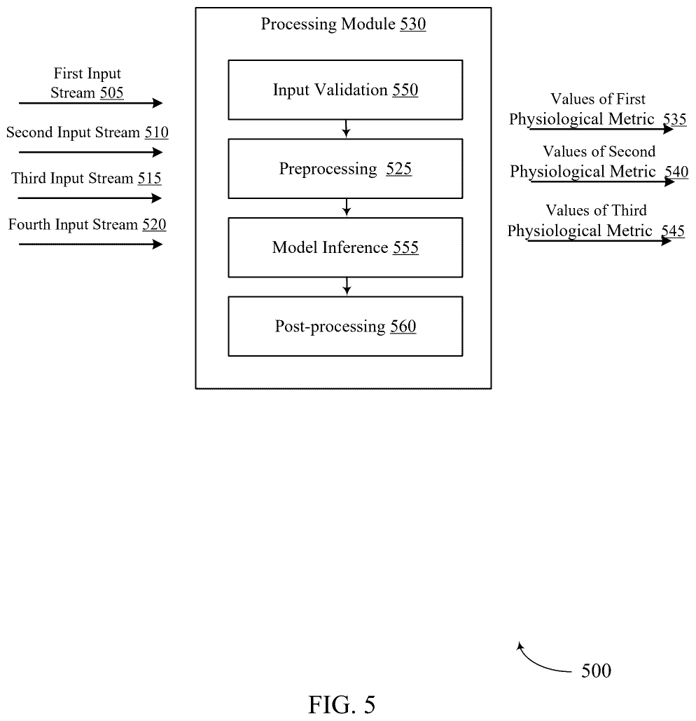

FIG. 5 shows an example of a system 500 that supports techniques for utilizing a multi-output neural network in accordance with aspects of the present disclosure. The system 500 may include a plurality of input streams, a processing module 530, and a plurality of outputs (e.g., values of characteristics), which may be examples of the input streams and values of characteristics as described with reference to FIGS. 1 through 4.

The processing module 530 may include an input validation block 550, a preprocessing block 525, a model inference block 555, and a post-processing block 560. The preprocessing block 525 may be an example of the preprocessing block 425 as described with reference to FIG. 4. The system 500 may be an example of the inference phase. The inference phase may correspond to the in-application implementation of the algorithm (e.g., including the multi-output neural network).

As described with reference to FIG. 4, the first input stream 505, the second input stream 510, the third input stream 515, the fourth input stream 520, or a combination thereof may be inputted into the processing module 530. The processing module 530 may output the values of the first physiological metric 535, the values of the second physiological metric 540, the values of the third physiological metric 545, or a combination thereof.

The processing module 530 may receive, at the input validation block 550, a validation after receiving the first input stream 505, the second input stream 510, the third input stream 515, the fourth input stream 520, or a combination thereof. The input validation block 550 may confirm that the processing module 530 received at least one of the first input stream 505, the second input stream 510, the third input stream 515, the fourth input stream 520, or a combination thereof. After confirming that the processing module 530 received at least one input stream, the system 500 may perform preprocessing operations at preprocessing block 525. The preprocessing block 525 may be an example of the preprocessing block 425, as described with reference to FIG. 4.

Using a single preprocessing block 525 for the input data (e.g., the first input stream 505, the second input stream 510, the third input stream 515, the fourth input stream 520, or a combination thereof) may enable the system 500 to operate at increased speeds and efficiency from utilizing the multi-output neural network. In some cases, the system 500 (e.g., including the user device) may perform the preprocessing procedures at preprocessing block 525 while operating the multi-output neural network in parallel. In other examples, the system 500 may perform the preprocessing procedures at preprocessing block 525 and then operate the multi-output neural network in series (e.g., perform the preprocessing procedures prior to operating the multi-output neural network).

After performing the preprocessing operations, the system 500 may perform operations at the model inference block 555. The model inference block 555 may implement the multi-output neural network into the application of the user device. In some cases, the system 500 may perform a post-processing procedure at the post-processing block 560 after implementing the multi-output neural network into the application of the user device. In such cases, the processing module 530 may output the values of the first physiological metric 535, the values of the second physiological metric 540, the values of the third physiological metric 545, or a combination thereof after performing the post-processing procedures at post-processing block 560. The processing module 530 may be an example of a single model file that includes matrix multiplication, preprocessing block 525 to encode data, post-processing block 560 to encode data, or a combination thereof.

Utilizing the multi-output neural network may allow the system 500 to include a single algorithm (e.g., framework) to handle several use-cases (e.g., multiple different characteristics). The single model may be maintained and knowledge may be transferred within the single model to improve future calculations and improve the overall efficiency and accuracy of the multi-output neural network. The computational time may be decreased by utilizing the multi-output neural network and several outputs (e.g., the values of the first physiological metric 535, the values of the second physiological metric 540, the values of the third physiological metric 545, or a combination thereof) may be returned at a same time as opposed to operating at least three individual models in parallel to generate the values of the characteristics.

In such cases, the system 500 may benefit from running a single model (e.g., utilizing the multi-output neural network) that operates at increased speeds and efficiency to generate and output multiple outputs. In other systems, operating multiple, individual models in parallel may decrease the efficiency of the overall system by performing separate, individual preprocessing and postprocessing procedures. However, by including a single preprocessing block 525 and single post-processing block 560 in system 500, multiple resources (e.g., including power consumption, execution time, and the like) may be saved to perform the pre and post processing steps.

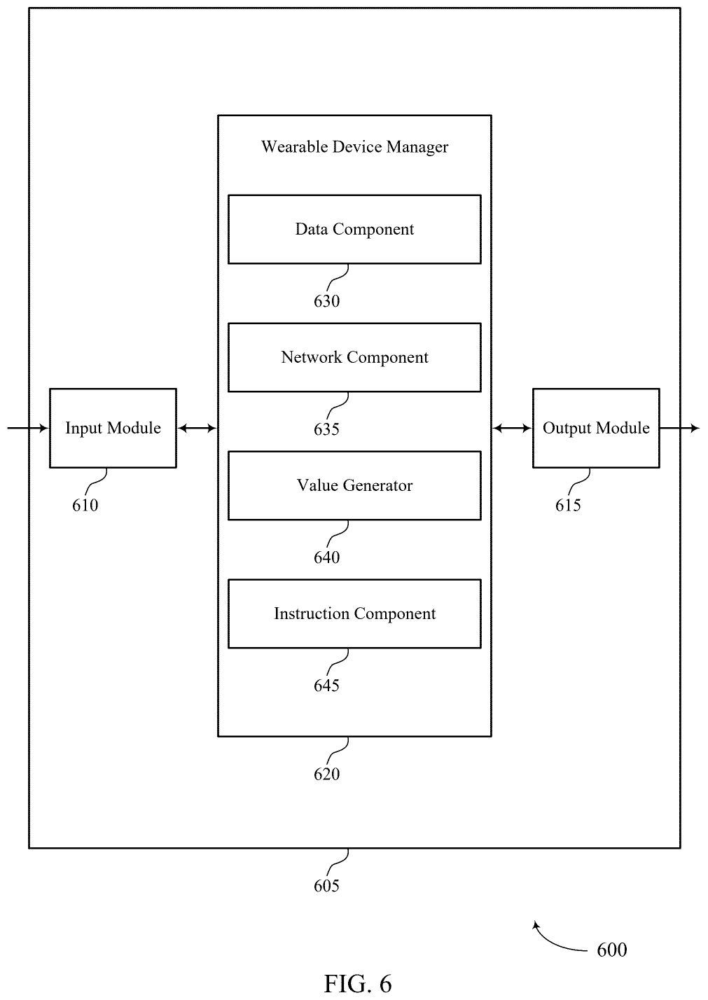

FIG. 6 shows a block diagram 600 of a device 605 that supports techniques for utilizing a multi-output neural network in accordance with aspects of the present disclosure. The device 605 may include an input module 610, an output module 615, and a wearable device manager 620. The device 605, or one of more components of the device 605 (e.g., the input module 610, the output module 615, the wearable device manager 620), may include at least one processor, which may be coupled with at least one memory, to support the described techniques. Each of these components may be in communication with one another (e.g., via one or more buses).

For example, the wearable device manager 620 may include a data component 630, a network component 635, a value generator 640, an instruction component 645, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The data component 630 may be configured as or otherwise support a means for receiving physiological data acquired via a wearable device via one or more electronic signals. The network component 635 may be configured as or otherwise support a means for inputting the physiological data into a multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both. The value generator 640 may be configured as or otherwise support a means for generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon. The instruction component 645 may be configured as or otherwise support a means for transmitting an instruction to a graphical user interface (GUI) of a user device associated with the wearable device, the instruction configured to cause the GUI to display one or more messages based at least in part on generating the one or more values associated with the first physiological metric and the one or more values associated with the second physiological metric.

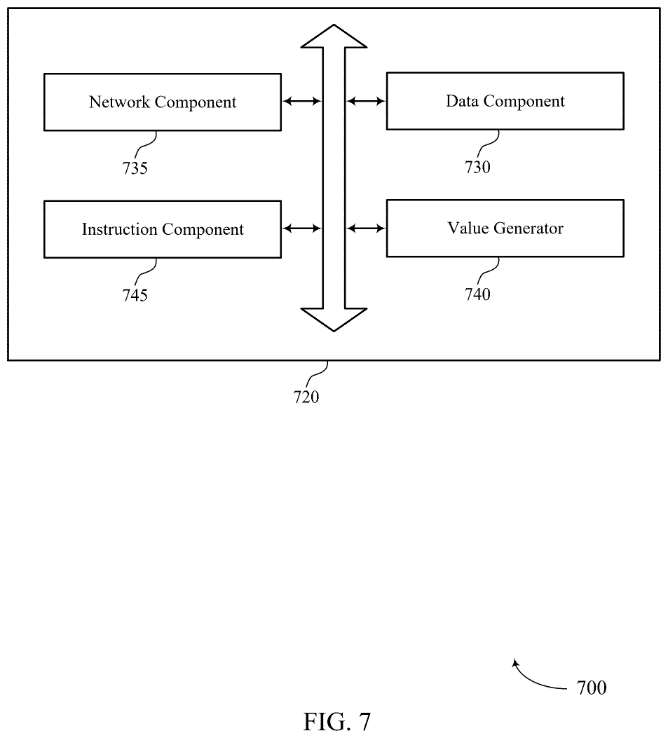

FIG. 7 shows a block diagram 700 of a wearable device manager 720 that supports techniques for utilizing a multi-output neural network in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of techniques for utilizing a multi-output neural network as described herein. For example, the wearable device manager 720 may include a data component 730, a network component 735, a value generator 740, an instruction component 745, or any combination thereof. Each of these components, or components of subcomponents thereof (e.g., one or more processors, one or more memories), may communicate, directly or indirectly, with one another (e.g., via one or more buses).

The data component 730 may be configured as or otherwise support a means for receiving physiological data acquired via a wearable device via one or more electronic signals. The network component 735 may be configured as or otherwise support a means for inputting the physiological data into a multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both. The value generator 740 may be configured as or otherwise support a means for generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon. The instruction component 745 may be configured as or otherwise support a means for transmitting an instruction to a GUI of a user device associated with the wearable device, the instruction configured to cause the GUI to display one or more messages based at least in part on generating the one or more values associated with the first physiological metric and the one or more values associated with the second physiological metric.

In some examples, the physiological data comprises a third input stream corresponding to temperature data. In some examples, the first physiological metric and the second physiological metric each comprises an input stream from at least one of the first input stream, the second input stream, the third input stream, or a combination thereof.

In some examples, the physiological data comprises a fourth input stream corresponding to blood oxygen data. In some examples, the first physiological metric and the second physiological metric each comprises an input stream from at least one of the first input stream, the second input stream, the third input stream, the fourth input stream, or a combination thereof.

In some examples, a training accuracy associated with training the multi-output neural network to compute the one or more values of the first physiological metric is increased based at least in part on the multi-output neural network being trained to compute the one or more values of the second physiological metric.

In some examples, the multi-output neural network is trained to simultaneously compute one or more values associated with a third physiological metric, wherein the third physiological metric comprises an input stream from at least one of the first input stream, the second input stream, or both. In some examples, the value generator 740 may be configured as or otherwise support a means for generating, via the single pass of the multi-output neural network, the one or more values associated with the third physiological metric, wherein the third physiological metric is associated with a third physiological phenomenon that is distinct from the first physiological phenomenon and the second physiological phenomenon.

In some examples, the network component 735 may be configured as or otherwise support a means for training the multi-output neural network based at least in part on inputting the physiological data into the multi-output neural network and generating the one or more values associated with the first physiological metric and the one or more values associated with the second physiological metric.

In some examples, the network component 735 may be configured as or otherwise support a means for training the multi-output neural network based on a plurality of features within a training physiological dataset associated with a plurality of users.

In some examples, the data component 730 may be configured as or otherwise support a means for processing the physiological data simultaneously prior to inputting the physiological data into the multi-output neural network.

In some examples, the first physiological metric is associated with a probability that the user experienced the first physiological phenomenon during a time period and the second physiological metric is associated with a probability that the user experienced the second physiological phenomenon during the time period.

In some examples, the one or more values associated with the first physiological metric comprises a first time scale and the one or more values associated with the second physiological metric comprise a second time scale same as the first time scale.

In some examples, the one or more values associated with the first physiological metric comprises a first resolution and the one or more values associated with the second physiological metric comprise a second resolution same as the first resolution.

In some examples, the physiological data is acquired throughout a time interval that includes one or more sleep intervals of the user.

In some examples, at least one of the first physiological metric and the second physiological metric comprises at least one of a breathing disturbance event, a sleep staging metric, a blood oxygen metric, a blood pressure metric, a heartbeat metric, or a respiratory rate metric.

In some examples, the wearable device comprises a wearable ring device.

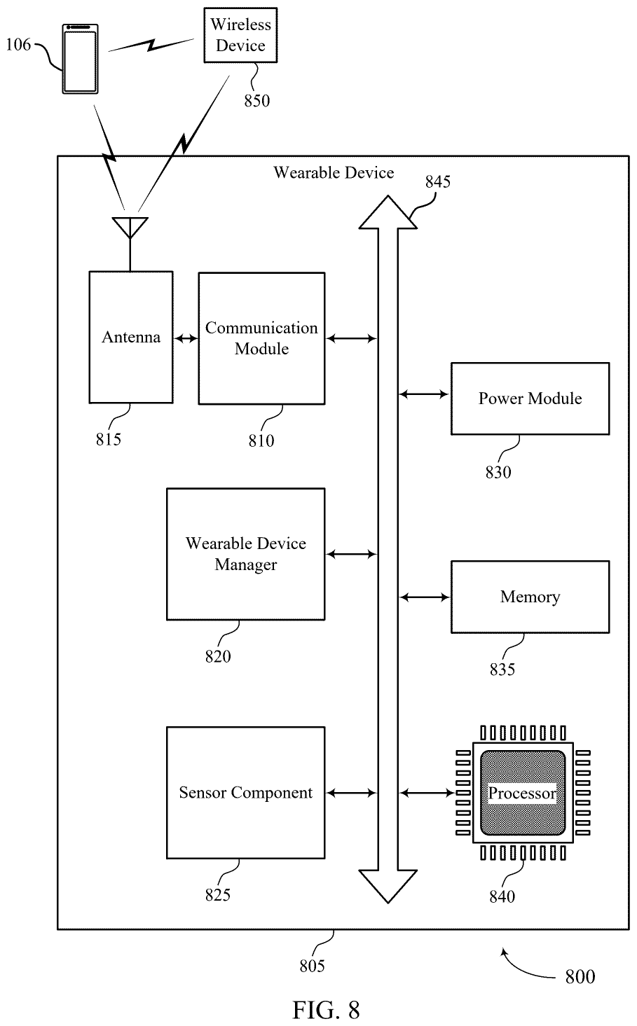

FIG. 8 shows a diagram of a system 800 including a device 805 that supports techniques for utilizing a multi-output neural network in accordance with aspects of the present disclosure. The device 805 may be an example of or include components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, one or more antennas 815, a sensor component 825, a power module 830, at least one memory 835, at least one processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for receiving physiological data acquired via a wearable device via one or more electronic signals. The wearable device manager 820 may be configured as or otherwise support a means for inputting the physiological data into a multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both. The wearable device manager 820 may be configured as or otherwise support a means for generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon. The wearable device manager 820 may be configured as or otherwise support a means for transmitting an instruction to a GUI of a user device associated with the wearable device, the instruction configured to cause the GUI to display one or more messages based at least in part on generating the one or more values associated with the first physiological metric and the one or more values associated with the second physiological metric.

By including or configuring the wearable device manager 820 in accordance with examples as described herein, the device 805 may support techniques for improved communication reliability, reduced latency, improved user experience related to reduced processing, reduced power consumption, more efficient utilization of communication resources, improved coordination between devices, longer battery life, and improved utilization of processing capability.

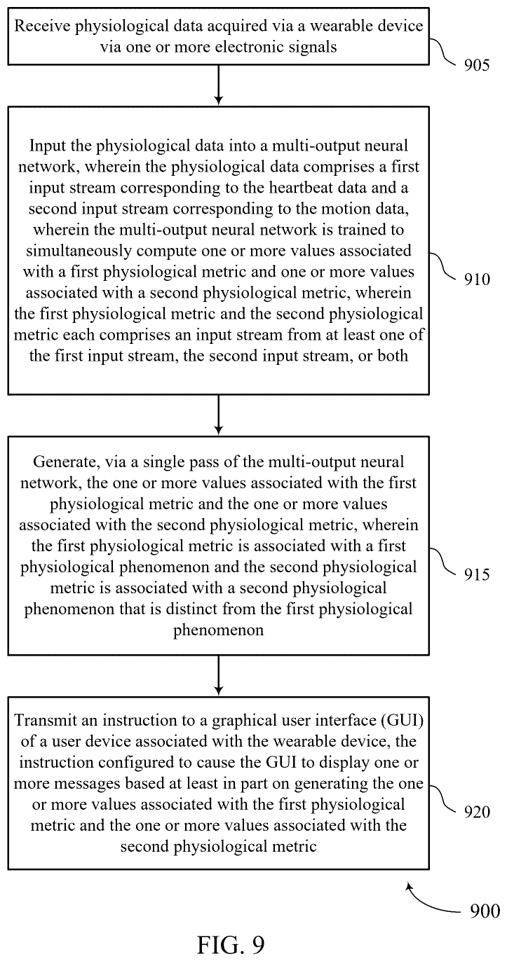

FIG. 9 shows a flowchart illustrating a method 900 that supports techniques for utilizing a multi-output neural network in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include receiving physiological data acquired via a wearable device via one or more electronic signals. The operations of 905 may be performed in accordance with examples as disclosed herein. In some examples, aspects of the operations of 905 may be performed by a data component 730 as described with reference to FIG. 7.

At 910, the method may include inputting the physiological data into a multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both. The operations of 910 may be performed in accordance with examples as disclosed herein. In some examples, aspects of the operations of 910 may be performed by a network component 735 as described with reference to FIG. 7.

At 915, the method may include generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon. The operations of 915 may be performed in accordance with examples as disclosed herein. In some examples, aspects of the operations of 915 may be performed by a value generator 740 as described with reference to FIG. 7.

At 920, the method may include transmitting an instruction to a graphical user interface (GUI) of a user device associated with the wearable device, the instruction configured to cause the GUI to display one or more messages based at least in part on generating the one or more values associated with the first physiological metric and the one or more values associated with the second physiological metric. The operations of 920 may be performed in accordance with examples as disclosed herein. In some examples, aspects of the operations of 920 may be performed by an instruction component 745 as described with reference to FIG. 7.

It should be noted that the methods described above describe possible implementations, and that the operations and the steps may be rearranged or otherwise modified and that other implementations are possible. Furthermore, aspects from two or more of the methods may be combined.

An apparatus (e.g., system for utilizing a multi-output neural network) is described. The apparatus include a wearable device configured to acquire physiological data from a user, the physiological data comprising heartbeat data collected via PPG measurements from one or more light-emitting components and one or more light-receiving components of the wearable device, motion data collected via one or more accelerometers of the wearable device, or both, one or more processors communicatively coupled with the wearable device, wherein the one or more processors are configured to receive the physiological data acquired via the wearable device via one or more electronic signals, input the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both, generate, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon, and transmit an instruction to a GUI of a user device associated with the wearable device, the instruction configured to cause the GUI to display one or more messages based at least in part on generating the one or more values associated with the first physiological metric and the one or more values associated with the second physiological metric.

In some examples of the apparatus described herein, the physiological data comprises a third input stream corresponding to temperature data and the first physiological metric and the second physiological metric each comprises an input stream from at least one of the first input stream, the second input stream, the third input stream, or a combination thereof.

In some examples of the apparatus described herein, the physiological data comprises a fourth input stream corresponding to blood oxygen data and the first physiological metric and the second physiological metric each comprises an input stream from at least one of the first input stream, the second input stream, the third input stream, the fourth input stream, or a combination thereof.

In some examples of the apparatus described herein, a training accuracy associated with training the multi-output neural network to compute the one or more values of the first physiological metric may be increased based at least in part on the multi-output neural network being trained to compute the one or more values of the second physiological metric.

In some examples of the apparatus described herein, the multi-output neural network is trained to simultaneously compute one or more values associated with a third physiological metric, wherein the third physiological metric comprises an input stream from at least one of the first input stream, the second input stream, or both and the apparatus may include further operations, features, means, or instructions for generating, via the single pass of the multi-output neural network, the one or more values associated with the third physiological metric, wherein the third physiological metric may be associated with a third physiological phenomenon that may be distinct from the first physiological phenomenon and the second physiological phenomenon.

Some examples of the apparatus described herein may further include operations, features, means, or instructions for training the multi-output neural network based at least in part on inputting the physiological data into the multi-output neural network and generating the one or more values associated with the first physiological metric and the one or more values associated with the second physiological metric.

Some examples of the apparatus described herein may further include operations, features, means, or instructions for training the multi-output neural network based on a plurality of features within a training physiological dataset associated with a plurality of users.

Some examples of the apparatus described herein may further include operations, features, means, or instructions for processing the physiological data simultaneously prior to inputting the physiological data into the multi-output neural network.

In some examples of the apparatus described herein, the first physiological metric may be associated with a probability that the user experienced the first physiological phenomenon during a time period and the second physiological metric may be associated with a probability that the user experienced the second physiological phenomenon during the time period.

In some examples of the apparatus described herein, the one or more values associated with the first physiological metric comprises a first time scale and the one or more values associated with the second physiological metric comprise a second time scale same as the first time scale.

In some examples of the apparatus described herein, the one or more values associated with the first physiological metric comprises a first resolution and the one or more values associated with the second physiological metric comprise a second resolution same as the first resolution.

In some examples of the apparatus described herein, the physiological data may be acquired throughout a time interval that includes one or more sleep intervals of the user.

In some examples of the apparatus described herein, at least one of the first physiological metric and the second physiological metric comprises at least one of a breathing disturbance event, a sleep staging metric, a blood oxygen metric, a blood pressure metric, a heartbeat metric, or a respiratory rate metric.

In some examples of the apparatus described herein, the wearable device comprises a wearable ring device.

A method by is described. The method may include receiving physiological data acquired via a wearable device via one or more electronic signals, inputting the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both, generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon, and transmitting an instruction to a GUI of a user device associated with the wearable device, the instruction configured to cause the GUI to display one or more messages based at least in part on generating the one or more values associated with the first physiological metric and the one or more values associated with the second physiological metric.

An apparatus is described. The apparatus may include one or more memories storing processor executable code, and one or more processors coupled with the one or more memories. The one or more processors may individually or collectively be operable to execute the code to cause the apparatus to receive physiological data acquired via a wearable device via one or more electronic signals, input the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both, generate, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon, and transmit an instruction to a GUI of a user device associated with the wearable device, the instruction configured to cause the GUI to display one or more messages based at least in part on generating the one or more values associated with the first physiological metric and the one or more values associated with the second physiological metric.

Another apparatus is described. The apparatus may include means for receiving physiological data acquired via a wearable device via one or more electronic signals, means for inputting the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both, means for generating, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon, and means for transmitting an instruction to a GUI of a user device associated with the wearable device, the instruction configured to cause the GUI to display one or more messages based at least in part on generating the one or more values associated with the first physiological metric and the one or more values associated with the second physiological metric.

A non-transitory computer-readable medium storing code is described. The code may include instructions executable by one or more processors to receive physiological data acquired via a wearable device via one or more electronic signals, input the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both, generate, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon, and transmit an instruction to a GUI of a user device associated with the wearable device, the instruction configured to cause the GUI to display one or more messages based at least in part on generating the one or more values associated with the first physiological metric and the one or more values associated with the second physiological metric.

In some examples of the method, apparatus, and non-transitory computer-readable medium described herein, the physiological data comprises a third input stream corresponding to temperature data and the first physiological metric and the second physiological metric each comprises an input stream from at least one of the first input stream, the second input stream, the third input stream, or a combination thereof.

In some examples of the method, apparatus, and non-transitory computer-readable medium described herein, the physiological data comprises a fourth input stream corresponding to blood oxygen data and the first physiological metric and the second physiological metric each comprises an input stream from at least one of the first input stream, the second input stream, the third input stream, the fourth input stream, or a combination thereof.

In some examples of the method, apparatus, and non-transitory computer-readable medium described herein, a training accuracy associated with training the multi-output neural network to compute the one or more values of the first physiological metric may be increased based at least in part on the multi-output neural network being trained to compute the one or more values of the second physiological metric.

In some examples of the method, apparatus, and non-transitory computer-readable medium described herein, the multi-output neural network is trained to simultaneously compute one or more values associated with a third physiological metric, wherein the third physiological metric comprises an input stream from at least one of the first input stream, the second input stream, or both. Some examples of the method, apparatus, and non-transitory computer-readable medium described herein may further include operations, features, means, or instructions for generating, via the single pass of the multi-output neural network, the one or more values associated with the third physiological metric, wherein the third physiological metric may be associated with a third physiological phenomenon that may be distinct from the first physiological phenomenon and the second physiological phenomenon.

Some examples of the method, apparatus, and non-transitory computer-readable medium described herein may further include operations, features, means, or instructions for training the multi-output neural network based at least in part on inputting the physiological data into the multi-output neural network and generating the one or more values associated with the first physiological metric and the one or more values associated with the second physiological metric.

Some examples of the method, apparatus, and non-transitory computer-readable medium described herein may further include operations, features, means, or instructions for training the multi-output neural network based on a plurality of features within a training physiological dataset associated with a plurality of users.

Some examples of the method, apparatus, and non-transitory computer-readable medium described herein may further include operations, features, means, or instructions for processing the physiological data simultaneously prior to inputting the physiological data into the multi-output neural network.

In some examples of the method, apparatus, and non-transitory computer-readable medium described herein, the first physiological metric may be associated with a probability that the user experienced the first physiological phenomenon during a time period and the second physiological metric may be associated with a probability that the user experienced the second physiological phenomenon during the time period.

In some examples of the method, apparatus, and non-transitory computer-readable medium described herein, the one or more values associated with the first physiological metric comprises a first time scale and the one or more values associated with the second physiological metric comprise a second time scale same as the first time scale.

In some examples of the method, apparatus, and non-transitory computer-readable medium described herein, the one or more values associated with the first physiological metric comprises a first resolution and the one or more values associated with the second physiological metric comprise a second resolution same as the first resolution.

In some examples of the method, apparatus, and non-transitory computer-readable medium described herein, the physiological data may be acquired throughout a time interval that includes one or more sleep intervals of the user.

In some examples of the method, apparatus, and non-transitory computer-readable medium described herein, at least one of the first physiological metric and the second physiological metric comprises at least one of a breathing disturbance event, a sleep staging metric, a blood oxygen metric, a blood pressure metric, a heartbeat metric, or a respiratory rate metric.

In some examples of the method, apparatus, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for utilizing a multi-output neural network, comprising:
a wearable device configured to acquire physiological data from a user, the physiological data comprising heartbeat data collected via photoplethysmogram (PPG) measurements from one or more light-emitting components and one or more light-receiving components of the wearable device, motion data collected via one or more accelerometers of the wearable device, or both; and
one or more processors communicatively coupled with the wearable device, wherein the one or more processors are configured to:
receive the physiological data acquired via the wearable device via one or more electronic signals;
input the physiological data into the multi-output neural network, wherein the physiological data comprises a first input stream corresponding to the heartbeat data and a second input stream corresponding to the motion data, wherein the multi-output neural network is trained to simultaneously compute one or more values associated with a first physiological metric and one or more values associated with a second physiological metric, wherein the first physiological metric and the second physiological metric each comprises an input stream from at least one of the first input stream, the second input stream, or both;
generate, via a single pass of the multi-output neural network, the one or more values associated with the first physiological metric and the one or more values associated with the second physiological metric, wherein the first physiological metric is associated with a first physiological phenomenon and the second physiological metric is associated with a second physiological phenomenon that is distinct from the first physiological phenomenon; and
transmit an instruction to a graphical user interface (GUI) of a user device associated with the wearable device, the instruction configured to cause the GUI to display one or more messages based at least in part on generating the one or more values associated with the first physiological metric and the one or more values associated with the second physiological metric.

2. The system of claim 1, wherein the physiological data comprises a third input stream corresponding to temperature data, wherein the first physiological metric and the second physiological metric each comprises an input stream from at least one of the first input stream, the second input stream, the third input stream, or a combination thereof.

3. The system of claim 2, wherein the physiological data comprises a fourth input stream corresponding to blood oxygen data, wherein the first physiological metric and the second physiological metric each comprises an input stream from at least one of the first input stream, the second input stream, the third input stream, the fourth input stream, or a combination thereof.

4. The system of claim 1, wherein a training accuracy associated with training the multi-output neural network to compute the one or more values of the first physiological metric is increased based at least in part on the multi-output neural network being trained to compute the one or more values of the second physiological metric.

5. The system of claim 1, wherein the multi-output neural network is trained to simultaneously compute one or more values associated with a third physiological metric, wherein the third physiological metric comprises an input stream from at least one of the first input stream, the second input stream, or both, wherein the one or more processors are further configured to:
generate, via the single pass of the multi-output neural network, the one or more values associated with the third physiological metric, wherein the third physiological metric is associated with a third physiological phenomenon that is distinct from the first physiological phenomenon and the second physiological phenomenon.

6. The system of claim 1, wherein the one or more processors are further configured to:
train the multi-output neural network based at least in part on inputting the physiological data into the multi-output neural network and generating the one or more values associated with the first physiological metric and the one or more values associated with the second physiological metric.

7. The system of claim 1, wherein the one or more processors are further configured to:
train the multi-output neural network based on a plurality of features within a training physiological dataset associated with a plurality of users.

8. The system of claim 1, wherein the one or more processors are further configured to:
process the physiological data simultaneously prior to inputting the physiological data into the multi-output neural network.

9. The system of claim 1, wherein the first physiological metric is associated with a probability that the user experienced the first physiological phenomenon during a time period and the second physiological metric is associated with a probability that the user experienced the second physiological phenomenon during the time period.

10. The system of claim 1, wherein the one or more values associated with the first physiological metric comprises a first time scale and the one or more values associated with the second physiological metric comprise a second time scale same as the first time scale.

11. The system of claim 1, wherein the one or more values associated with the first physiological metric comprises a first resolution and the one or more values associated with the second physiological metric comprise a second resolution same as the first resolution.

12. The system of claim 1, wherein the physiological data is acquired throughout a time interval that includes one or more sleep intervals of the user.

13. The system of claim 1, wherein at least one of the first physiological metric and the second physiological metric comprises at least one of a breathing disturbance event, a sleep staging metric, a blood oxygen metric, a blood pressure metric, a heartbeat metric, or a respiratory rate metric.

14. The system of claim 1, wherein the wearable device comprises a wearable ring device.
